(19) United States
(12) Patent Application Publication
(10) Pub. No.: US 2018/0235469 A1
(43) Pub. Date: Aug. 23, 2018

(54) VITAL SIGN MONITORING APPARATUS

(71) Applicant: NIHON KOHDEN CORPORATION, Tokyo (JP)

(72) Inventor: Kazuyoshi KITAHARA, Shinjuku-ku, Tokyo (JP)

(73) Assignee: NIHON KOHDEN CORPORATION, Tokyo (JP)

(21) Appl. No.: 15/751,250

(22) PCT Filed: Aug. 9, 2016
(86) PCT No.: PCT/JP2016/003689

§ 371 Date: Feb. 8, 2018

(30) Foreign Application Priority Data
Aug. 11, 2015 (JP) 2015-158854

(51) Int. Cl. A61B 5/00; A61B 5/0205; A61B 5/01; A61B 5/11

(52) U.S. Cl. CPC A61B 5/0015; A61B 5/0205; A61B 5/01; A61B 2562/0219; A61B 5/1117; A61B 2560/0209; A61B 5/7405

(57) ABSTRACT

A vital sign monitoring apparatus is configured to measure a vital sign of a subject. The vital sign monitoring apparatus includes a wireless communication section configured to transmit and to receive various data including audio data, and a determining section configured to determine whether a given condition is met and to switch the wireless communication section from a power saving mode to a high performance mode when it is determined that the given condition is met. In the power saving mode, a processing related to the audio data is partially restricted. In the high performance mode, the restriction of the processing related to the audio data is removed.

### TECHNICAL FIELD

The present invention relates to a vital sign monitoring apparatus.

### BACKGROUND ART

In recent years, vital sign monitoring apparatuses are widely used in hospitals to measure vital signs of a subject (patients), such as blood pressure, body temperature and respiration. The vital sign monitoring apparatuses include bedside monitors, medical telemeters and the like.

Some vital sign monitoring apparatuses are used not only for the purpose of measuring vital signs of a subject but also as a communication tool between the subject and a doctor or a nurse at a remote location. For example, JP2011-212167A discloses a vital sign monitoring apparatus configured to transmit a sound collected through a microphone to an external device. U.S. Pat. No. 8,487,771B2 discloses a personal health management device in which an audio communication technique is implemented.

However, audio communication functions require large power consumption (battery power consumption). Therefore, it is not desirable to keep an audio communication function turned on in portable type medical devices. The patent documents cited above do not give any teachings or suggestions regarding a power consumption of audio communication functions.

### SUMMARY

Illustrative aspects of the present invention provide a vital sign monitoring apparatus by which audio communication can be attained smoothly while power consumption is suppressed.

According to an illustrative aspect of the present invention, a vital sign monitoring apparatus is configured to measure a vital sign of a subject. The vital sign monitoring apparatus includes a wireless communication section configured to transmit and to receive various data including audio data, and a determining section configured to determine whether a given condition is met and to switch the wireless communication section from a power saving mode to a high performance mode when it is determined that the given condition is met. In the power saving mode, a processing related to the audio data is partially restricted. In the high performance mode, the restriction of the processing related to the audio data is removed.

### BRIEF DESCRIPTION OF DRAWINGS

FIG. 1 is a block diagram illustrating a configuration of a vital sign monitoring apparatus according to an exemplary embodiment of the present invention.

FIG. 2 is a table showing an example of conditions used in a determining section of the vital sign monitoring apparatus.

FIG. 3 is a flow chart of steps to be carried out by the determining section.

FIG. 4 is a table showing another example of conditions used in the determining section.

FIG. 5 is a flow chart of other steps to be carried out by the determining section.

### DESCRIPTION OF EMBODIMENTS

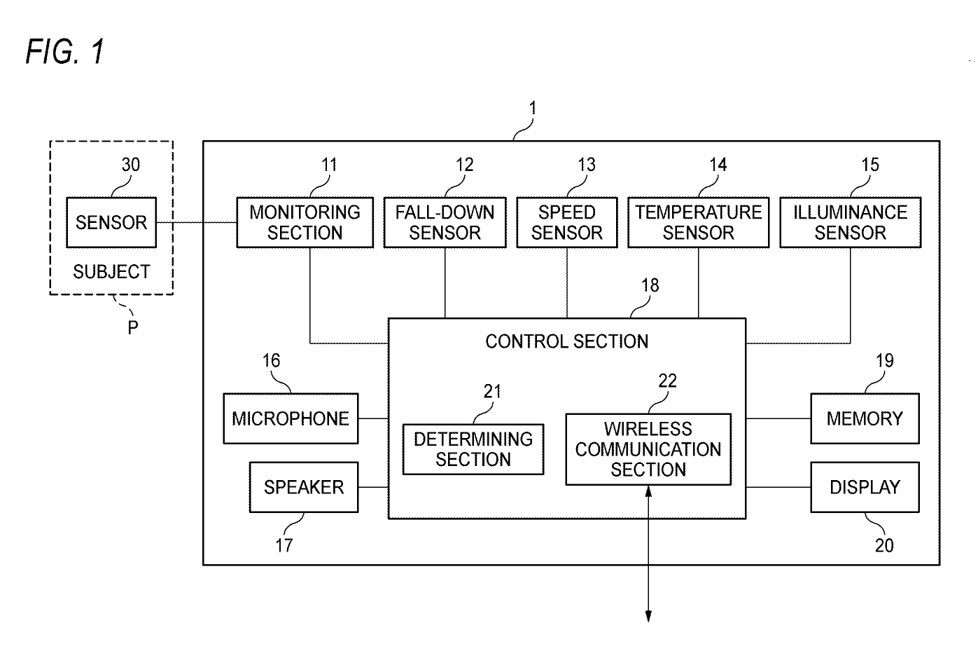

Hereinafter, exemplary embodiments of the present invention will be described with reference to the drawings. FIG. 1 is a block diagram illustrating a configuration of a vital sign monitoring apparatus 1 according to one exemplary embodiment of the present invention. The vital sign monitoring apparatus 1 is configured to obtain various vital signs (such as blood pressure, body temperature, a breathing waveform, the number of times of respiration, and arterial oxygen saturation) of a subject P. For example, the vital sign monitoring apparatus 1 is a medical telemeter carried by the subject P.

The vital sign monitoring apparatus 1 has a monitoring section 11, a fall-down sensor 12, a speed sensor 13, a temperature sensor 14, an illuminance sensor 15, a microphone 16, a speaker 17, a control section 18, a memory 19, and a display 20. The control section 18 has a determining section 21 and a wireless communication section 22. Moreover, although not shown, a battery, various control circuits etc. are also built in the vital sign monitoring apparatus 1.

Various sensors 30 are attached to the subject P to obtain vital signs. The sensors 30 include, for example, a probe, electrodes, and/or a cuff. The monitoring section 11 is configured to receive biological signals of the subject P from the sensors 30, and to obtain measured values of the vital signs from the biological signals. The monitoring section 11 sends the measured values of the vital signs to the control section 18.

The fall-down sensor 12 is configured to detect falling-down of the subject P to be monitored by the vital sign monitoring apparatus 1. For example, the fall-down sensor 12 may detect the falling-down by a geomagnetic field measuring function, and a gyro sensor may be applied to the fall-down sensor 12 to detect the falling-down. The fall-down sensor 12 sends notification to the control section 18 when the falling-down of the subject P is detected.

The speed sensor 13 is configured to detect movement speed of the vital sign monitoring apparatus 1. Any sensor may be used as the speed sensor 13 as long as it has a general speed detecting mechanism. The speed sensor 13 supplies the detected speed to the control section 18 suitably. The speed sensor 13 may be an acceleration sensor and may be configured to calculate acceleration rather than the movement speed.

The temperature sensor 14 is configured to measure an ambient temperature around the vital sign monitoring apparatus 1. Any sensor may be used as the temperature sensor 14 as long as it has a similar configuration to a general thermometer etc. The temperature sensor 14 supplies a detected temperature to the control section 18 suitably.

The illuminance sensor 15 is configured to measure ambient illuminance around the vital sign monitoring apparatus 1. Any sensor can be used as the illuminance sensor 15 as long as it has a general illuminance measuring mechanism (which is, for example, configured to have a photodiode, a current amplifier circuit etc.). The illuminance sensor 15 sends a measured value of the detected ambient illuminance to the control section 18 as necessary.

The vital sign monitoring apparatus 1 may not include the fall-down sensor 12, the speed sensor 13, the temperature sensor 14 and the illuminance sensor 15. The vital sign monitoring apparatus 1 may include only necessary one or more of the sensors 12, 13, 14, 15. The vital sign monitoring apparatus 1 may include other sensor(s) not illustrated in the drawings, such as a vibration sensor.

The microphone 16 is a sound collecting device configured to collect an ambient sound around the vital sign monitoring apparatus 1. The microphone 16 sends the collected sound to the control section 18 as necessary. The speaker 17 outputs a sound such as a notification sound in accordance with control of the control section 18.

The memory 19 includes a non-volatile memory (e.g., a hard disk) storing various data, and a volatile memory (e.g., a cache memory) used for data buffering or as a work area. The control section 18 reads/writes data from/onto the memory 19 suitably.

The display 20 is provided on a housing of the vital sign monitoring apparatus 1. The display 20 is, for example, a liquid crystal display, and may include its peripheral devices. Information about vital signs (e.g., measured values and/or measured waveforms of blood pressure, breathing, body temperature and/or arterial oxygen saturation) of the subject P is displayed on the display 20.

The control section 18 performs various kinds of control on the vital sign monitoring apparatus 1. The control section 18 includes a central processing unit (CPU) and various circuits. The CPU reads various programs from the memory 19 and executes the read programs. The control section 18 may include a plurality of CPUs, and may be implemented by a configuration having a plurality of chips.

The control section 18 has the determining section 21 and the wireless communication section 22. The wireless communication section 22 transmits/receives various data including audio data to/from another apparatus (e.g., a central monitor). A technique for transferring audio data in real time is implemented in the wireless communication section 22. More specifically, the wireless communication section 22 has a voice over Internet protocol (VoIP) function to encode/encrypt audio data and to transmit/receive the encoded/encrypted audio data. Here, the audio data means any data of sound which may include conversations.

The wireless communication section 22 has two operating modes, that is, a high performance mode and a power saving mode. The high performance mode is a mode in which the VoIP function is turned on (i.e. encoding or encryption for audio transmission and reception can be executed). That is, in the high performance mode, the restriction of the processing related to the audio data is removed (the function of real-time transmission of audio data is available). In the high performance mode, the transmission and reception of the audio data can be executed accurately but power consumption (power consumption of a battery built in the vital sign monitoring apparatus 1) is large.

The power saving mode is a mode in which at least a part of the processing related to the audio data is restricted (i.e. at least a part of the encoding or encryption for audio transmission and reception cannot be executed). In the power saving mode, the audio data can be received from an external device but a processing such as a decoding of the audio data is restricted. That is, in the power saving mode, real-time transmission of the audio data in compliance with the existing protocol (VoIP protocol) is not available but the power consumption is small.

The operating modes (the high performance mode and the power saving mode) of the wireless communication section 22 can be changed from one to the other in accordance with control of the determining section 21. A determination process of the determining section 21 will be described below with reference to an example.

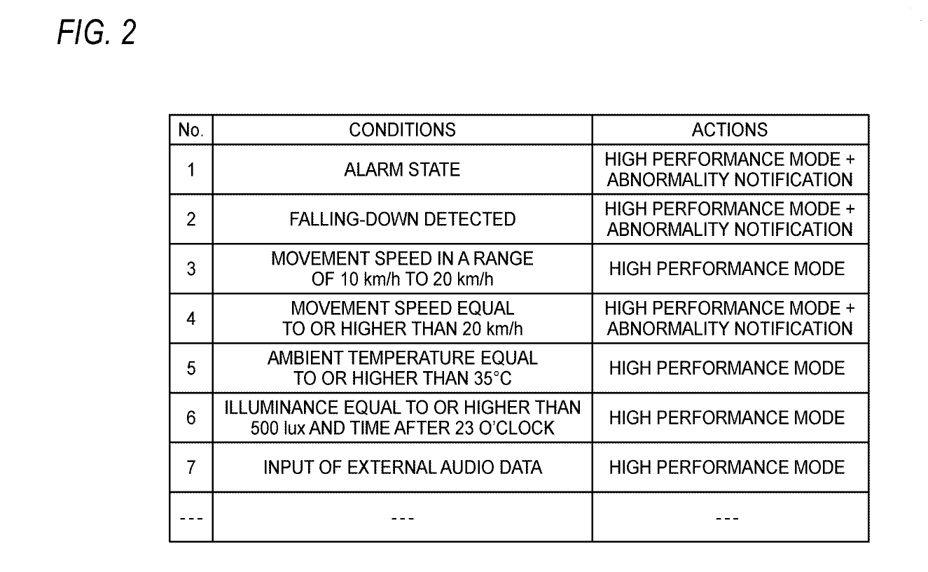

First, a control process of the determining section 21 in the case where the wireless communication section 22 is operating in the power saving mode will be described. The determining section 21 determines whether any of given conditions is met. The given conditions are various conditions used when switching the mode from the power saving mode to the high performance mode. FIG. 2 is a table showing examples of the given conditions which are used when the wireless communication section 22 is operating in the power saving mode. FIG. 2 shows seven examples of conditions.

The determining section 21 switches the wireless communication section 22 to operate in the high performance mode when a measured value of a vital sign received from the monitoring section 11 becomes outside a normal range, i.e. when it is in a state in which an alarm should be turned on (an alarm state) (No. 1 in FIG. 2). Depending on conditions, the determining section 21 may send a notification to an external device (e.g., to a central server and/or to a mobile device carried by a nurse). When, for example, the subject P is in the alarm state, the determining section 21 determines that a notification be sent to a mobile device of a nurse. The wireless communication section 22 may then send out a notification that the subject P is in an alarm state and the details of the state of the subject. The notification may be an audio notification or a text notification.

When falling-down is detected by the fall-down sensor 12, the determining section 21 switches the wireless communication section 22 to operate in the high performance mode and sends a notification to the external device that the subject P has fallen down (No. 2 in FIG. 2).

When the movement speed detected by the speed sensor 13 is in a range of 10 km/h to 20 km/h (within a given speed range), the determining section 21 switches the wireless communication section 22 to operate in the high performance mode (No. 3 in FIG. 2).

When the movement speed detected by the speed sensor 13 is equal to or higher than 20 km/h (higher than the given speed range), the determining section 21 switches the wireless communication section 22 to operate in the high performance mode and sends a notification (a notification that the subject P is in an abnormal state such as a state in which the subject P is running) to the external device (No. 4 in FIG. 2).

When the ambient temperature detected by the temperature sensor 14 is equal to or higher than 35° C., the determining section 21 switches the wireless communication section 22 to operate in the high performance mode (No. 5 in FIG. 2). When the ambient illuminance detected by the illuminance sensor 15 is equal to or higher than 500 lux and the time is after 23 o'clock, the determining section 21 switches the wireless communication section 22 to operate in the high performance mode (No. 6 in FIG. 2). When the wireless communication section 22 receives audio data from the external device (e.g., the central server or the mobile device carried by the nurse), the determining section 21 switches the wireless communication section 22 to operate in the high performance mode (No. 7 in FIG. 2).

The conditions shown in FIG. 2 are merely examples. The vital sign monitoring apparatus 1 may be configured to allow a user (e.g., a doctor or a nurse) to optionally set conditions and actions corresponding to the conditions.

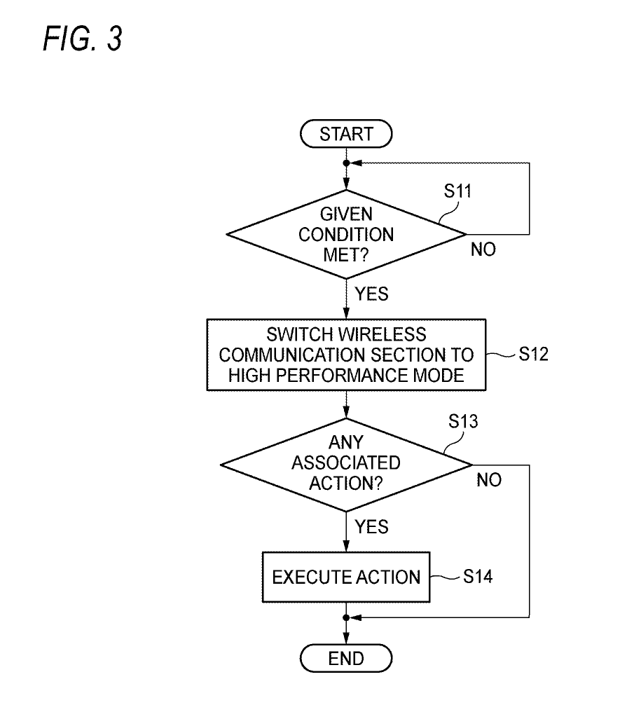

FIG. 3 is a flow chart showing operations of the determining section 21 when the wireless communication section 22 is operating in the power saving mode. The determining section 21 periodically determines whether any of the given conditions (FIG. 2) is met (S11). When none of the condition is met (S11: No), the determining section 21 continuously makes condition determination.

On the other hand, when any of the given conditions is met (S11: Yes), the determining section 21 switches the wireless communication section 22 to operate in the high performance mode (S12). The determining section 21 determines whether an action such as a notification is set correspondingly to the condition or not (S13). Particularly when nothing is set (S13: No), the determining section 21 terminates the processing. On the other hand, when an action such as a notification is set correspondingly to the condition that has been met (S13: Yes), the determining section 21 causes the wireless communication section 22 to execute the action (S14).

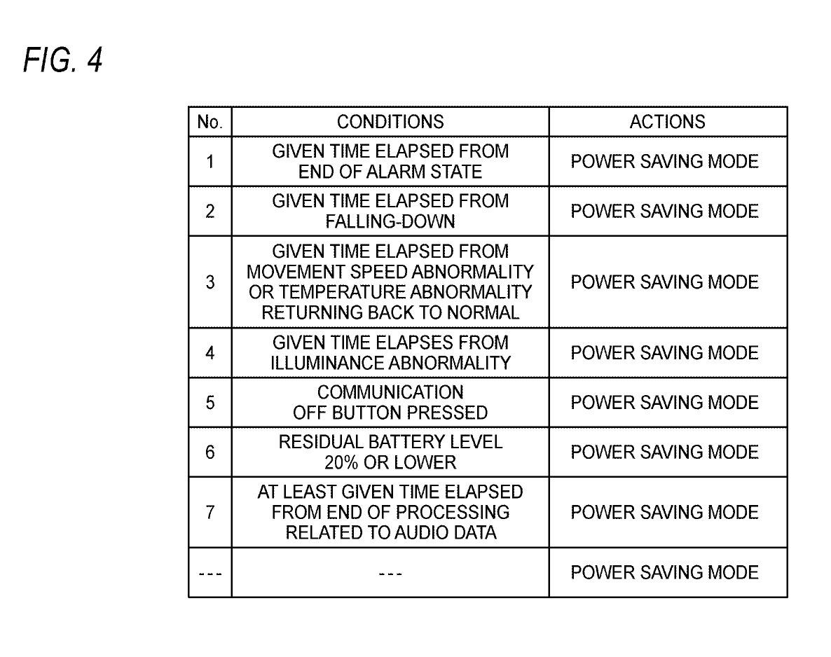

Next, a control process of the determining section 21 when the wireless communication section 22 is operating in the high performance mode will be described. FIG. 4 is a table showing examples of conditions used when the wireless communication section 22 is operating in the high performance mode. They are deactivating conditions defining conditions where the high performance mode should be deactivated.

When a given time elapses after a measured value of a vital sign received from the monitoring section 11 returns from a state in which an alarm should be turned on (the alarm state) to a normal state (No. 1 in FIG. 4), the determining section 21 switches the wireless communication section 22 to operate the power saving mode. Similarly, when a given time elapses after the fall-down sensor 12 detects falling-down, the determining section 21 switches the wireless communication section 22 to operate in the power saving mode (No. 2 in FIG. 4). Moreover, when a given time elapses after an abnormality (movement speed abnormality, temperature abnormality and/or illuminance abnormality) returns back to normal, the determining section 21 switches the wireless communication section 22 to operate in the power saving mode (No. 3 or No. 4 in FIG. 4). The determining section 21 may switch the wireless communication section 22 to operate in the power saving mode immediately after the abnormality returns back to normal. That is, the determining section 21 switches the wireless communication section 22 to operate in the power saving mode when an abnormality returns back to normal.

In addition, when it is detected that, for example, a button (audio communication end button) provided on the housing of the vital sign monitoring apparatus 1 is pushed down, the determining section 21 may switch the wireless communication section 22 to operate in the power saving mode (No. 5 in FIG. 4). Further, when the residual level of the battery of the vital sign monitoring apparatus 1 becomes 20% or lower, the determining section 21 may switch the wireless communication section 22 to operate in the power saving mode (No. 6 in FIG. 4). The threshold of the residual level of the battery is not limited to 20%, and may be a value other than 20%. In addition, when the processing related to the audio data is not performed for at least a given period of time, the determining section 21 switches the wireless communication section 22 to operate in the power saving mode (No. 7 in FIG. 4).

The conditions shown in FIG. 4 are merely examples. The vital sign monitoring apparatus 1 may be configured to allow a user (e.g., a doctor or a nurse) to optionally set conditions and actions corresponding to the conditions.

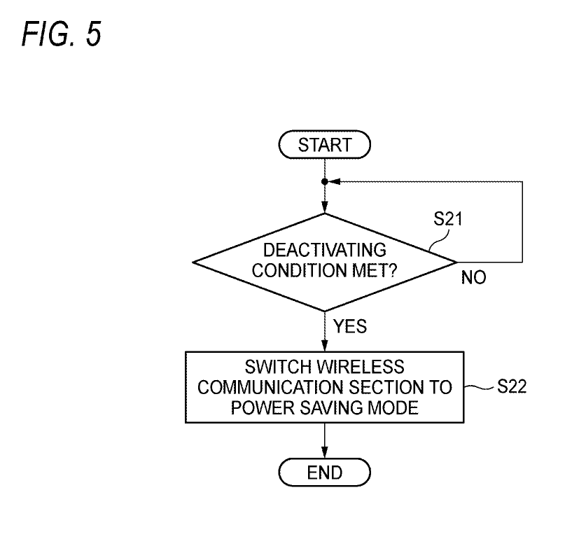

FIG. 5 is a flow chart illustrating operations of the determining section 21 in a case in which the wireless communication section 22 is operating in the high performance mode. The determining section 21 periodically determines whether one of the deactivating conditions shown in FIG. 4 is met (S21). When none of the deactivating conditions are met (S21: No), the determining section 21 continues the determining step. When one of the deactivating conditions is met (S21: Yes), the determining section 21 switches the wireless communication section 22 from the high performance mode to the power saving mode (S22).

The determining section 21 may perform various processes (display of a message for notifying of the mode change to the power saving mode etc.) suitably after the mode is changed to the power saving mode.

In the following, advantages of the vital sign monitoring apparatus 1 according to the exemplary embodiment will be described. As described above, the determining section 21 switches the wireless communication section 22 to operate in the high performance mode only when one or more conditions (e.g., one or more of the conditions shown in FIG. 2) is met. That is, the wireless communication section 22 is operated in the high performance mode in which the restriction of the processing related to the audio data is removed, only when it is deemed necessary. For example, encoding related to audio communication can be performed in the high performance mode. Otherwise, the wireless communication section 22 is operated in the power saving mode in which the processing related to the audio data is partially restricted, i.e., only limited functions such as text transmission and reception and audio data reception are implemented. Thus, power consumption of the wireless communication section 22 can be reduced. Hence, reduction in power consumption of the vital sign monitoring apparatus 1 can be achieved. In addition, in a situation in which a subject P and someone such as a doctor need to communicate, the processing related to the audio data is not restricted so that audio communication is available. Thus, the subject P can smoothly communicate with someone such as a doctor in case of emergency.

When, for example, a measured value of a vital sign of the subject P becomes outside a normal range, i.e. a condition in which an alarm is turned (No. 1 in FIG. 2), the subject P can attain audio communication through the microphone 16 and the speaker 17. Thus, the subject P can explain his/her body condition to the doctor etc. In addition, the doctor etc. can respond to that quickly.

In addition, when the falling-down of the subject P is detected, the subject P can attain audio communication through the microphone 16 and the speaker 17 (No. 2 in FIG. 2). Thus, the subject P can explain his/her condition to the doctor etc. In addition, the doctor etc. can respond to that quickly.

When the subject P is running (the movement speed is higher than a predetermined value), the vital sign monitoring apparatus 1 carried by the subject P changes to a state in which audio communication is enabled (No. 3 or No. 4 in FIG. 2). Thus, the doctor etc. can make conversation with the subject P to call his/her attention.

In addition, when the ambient temperature around the subject P is high (the ambient temperature is higher than a predetermined value), the vital sign monitoring apparatus 1 carried by the subject P changes to a state in which audio communication is enabled (No. 5 in FIG. 2). Thus, the doctor etc. can make conversation with the subject P to call his/her attention (e.g., conversation to urge the subject P to use an air conditioner). Further, when the ambient temperature around the subject P is remarkably high (e.g., the ambient temperature is higher than 50°), the vital sign monitoring apparatus 1 carried by the subject P changes to a state in which audio communication is enabled. At the same time, the vital sign monitoring apparatus 1 may send alarm notification. Thus, it is possible to promptly detect an incident such as a fire.

When the ambient illuminance around the subject P is high in a late-night time zone, the vital sign monitoring apparatus 1 carried by the subject P changes to a state in which audio communication is enabled (No. 6 in FIG. 2). Thus, the doctor etc. can make conversation with the subject P to call his/her attention (e.g., conversation to urge the subject P to sleep soon).

In addition, when one or more of deactivating conditions (e.g., the conditions shown in FIG. 4) is met, the determining section 21 switches the wireless communication section 22 from the high performance mode to the power saving mode. That is, when there appears a situation that audio communication is no longer necessary, the determining section 21 switches the wireless communication section 22 to operate in the power saving mode requiring lower battery power consumption. Thus, it is possible to suitably suppress power consumption of the battery built in the vital sign monitoring apparatus 1.

When, for example, it is thought that the abnormal state has been removed (No. 1 to No. 4 in FIG. 4), the determining section 21 switches the wireless communication section 22 from the high performance mode to the power saving mode. Thus, it is possible to avoid a situation that the high performance mode requiring large battery power consumption still continues.

In addition, when the subject P explicitly turns OFF audio communication (No. 5 in

FIG. 4), the determining section 21 switches the wireless communication section 22 from the high performance mode to the power saving mode. Thus, it is possible to suppress battery power consumption in accordance with the will of the subject P.

In addition, when the residual battery level is not higher than a predetermined value (e.g., 20%) (No. 6 in FIG. 4), the determining section 21 may forcibly switch the wireless communication section 22 from the high performance mode to the power saving mode. Generally, it is desirable that the vital signs be measured continuously by the vital sign monitoring apparatus 1. When the residual battery level is low, the processing on audio data which requires large power consumption is restricted so that the vital sign monitoring apparatus 1 can measure the vital signs for a long term.

When the processing related to the audio data is not performed for at least a given period of time (No. 7 in FIG. 4), the determining section 21 switches the wireless communication section 22 from the high performance mode to the power saving mode. Thus, it is possible to suppress unnecessary battery power consumption.

While the present invention has been described with reference to certain exemplary embodiments thereof, the scope of the present invention is not limited to the exemplary embodiments described above, and it will be understood by those skilled in the art that various changes and modifications may be made therein without departing from the scope of the present invention as defined by the appended claims.

At least a part of the processings of the monitoring section 11 and the control section 18 may be implemented as a computer program executable in the vital sign monitoring apparatus 1. In addition, a part of the processings of the various sensors (the fall-down sensor 12, the speed sensor 13, the temperature sensor 14 and the illuminance sensor 15) can be also implemented as the computer program executable in the vital sign monitoring apparatus 1.

The program may be stored in a non-transitory computer readable medium to be executed by a computer. The non-transitory computer readable medium includes various types of tangible storage medium. Examples of the non-transitory computer readable medium include magnetic recording medium (e.g., a flexible disk, a magnetic tape, and a hard disk drive), a magneto-optical recording medium (e.g., a magneto-optical disk), a CD-read only memory (CD-ROM), a CD-R, a CD-R/W, semiconductor memories (e.g., a mask ROM, a programmable ROM (PROM), an erasable PROM (EPROM), a flash ROM, and a random access memory (RAM)). Alternatively, the program may be executed on a computer by means of a transitory computer readable medium. Examples of the transitory computer readable medium include electrical signals, optical signals and electromagnetic waves. The transitory computer readable medium may provide the program to be executed on a computer through a wired communication such as an electric wire or an optical fiber or through a wireless communication.

This application is based on Japanese Patent Application No. 2015-158854 filed on Aug. 11, 2015, the entire content of which is incorporated herein by reference.

1. A vital sign monitoring apparatus configured to measure a vital sign of a subject, the vital sign monitoring apparatus comprising:
a wireless communication section configured to transmit and to receive various data including audio data; and
a determining section configured to determine whether a given condition is met and to switch the wireless communication section from a power saving mode to a high performance mode when it is determined that the given condition is met, wherein a processing related to the audio data is partially restricted in the power saving mode, and the restriction of the processing related to the audio data is removed in the high performance mode.

2. The vital sign monitoring apparatus according to claim 1, further comprising a monitoring section configured to receive biological signals from a sensor attached to the subject and to obtain a measured value of a vital sign,
wherein the determining section is configured to switch the wireless communication section from the power saving mode to the high performance mode when the measured value obtained by the monitoring section becomes outside a given range.

3. The vital sign monitoring apparatus according to claim 1, further comprising a fall-down sensor configured to detect a falling-down of the subject,
wherein the determining section is configured to switch the wireless communication section from the power saving mode to the high performance mode when the fall-down sensor detects the falling-down of the subject.

4. The vital sign monitoring apparatus according to claim 1, further comprising a speed sensor configured to detect a movement speed of the subject,
wherein the determining section is configured to switch the wireless communication section from the power saving mode to the high performance mode when the movement speed detected by the speed sensor is equal to or higher than a given speed.

5. The vital sign monitoring apparatus according to claim 1, wherein, when the wireless communication section is switched from the power saving mode to the high performance mode, the wireless communication section sends a notification to an external device.

6. The vital sign monitoring apparatus according to claim 1, wherein the determining section is configure to determine whether a deactivating condition is met when the wireless communication section is operating in the high performance mode, the deactivating condition defining a condition where the high performance mode should be deactivated, and when it is determined that the deactivating condition is met, the determining section switches the wireless communication section from the high performance mode to the power saving mode.

7. The vital sign monitoring apparatus according to claim 2, wherein the determining section is configured to switch the wireless communication section from the high performance mode to the power saving mode when the measured value obtained by the monitoring section and outside the normal range becomes within the given range.

8. The vital sign monitoring apparatus according to claim 1, wherein the determining section is configured to switch the wireless communication section from the high performance mode to the power saving mode when a residual level of a battery built in the vital sign monitoring apparatus is equal to or lower than a threshold.

9. The vital sign monitoring apparatus according to claim 1, wherein the determining section is configured to switch the wireless communication section from the high performance mode to the power saving mode when the processing related to the audio data is not performed for at least a given period of time.

10. (canceled)

11. A non-transitory computer readable medium storing a program that, when executed by a computer in a vital sign monitoring apparatus configured to measure a vital sign of a subject, causes the computer to execute a method comprising:
wirelessly transmitting and receiving various data including audio data;
determining whether a given condition is met; and
switching a mode from a power saving mode to a high performance mode when it is determined that the given condition is met, wherein a processing related to the audio data is partially restricted in the power saving mode, and the restriction of the processing related to the audio data removed in the high performance mode.
